(19) United States
(12) Patent Application Publication
(10) Pub. No.: US 2010/0049167 A1
(43) Pub. Date: Feb. 25, 2010

(54) INTRODUCER SHEATH HAVING REINFORCED DISTAL TAPER

(75) Inventor: Randy Joe Myers, Bloomington, IN (US)

Correspondence Address: BRINKS HOFER GILSON & LIONE/INDY/COOK;BRINKS HOFER GILSON & LIONE, CAPITAL CENTER, SUITE 1100, 201 NORTH ILLINOIS STREET, INDIANAPOLIS, IN, 46204-4220, US

(73) Assignee: Cook Incorporated, Bloomington, IN (US)

(21) Appl. No.: 12/194,717

(22) Filed: Aug. 20, 2008

(51) Int. Cl. A61M 25/01; B29C 65/00

(52) U.S. Cl. 604/527; 156/196

(57) ABSTRACT

An introducer sheath has an inner liner having a first length comprising a major diameter, a second length comprising a minor diameter, and a tapered third length axially disposed between the first and second lengths. A coil is positioned over the inner liner and spans at least a portion of each of the first, second, and third lengths. An outer jacket is positioned longitudinally around the reinforcing member and the inner liner, and is bonded to the outer surface of the inner liner. The outer jacket has a first length comprising a major diameter, a second length comprising a minor diameter, and a tapered third length axially disposed between the first and second lengths. The outer diameter first, second, and third lengths substantially correspond to and are disposed radially outwardly of the respective inner liner first, second, and third lengths.

### BACKGROUND

1. Technical Field

This invention relates to the field of medical introducer apparatuses. More particularly, the invention relates to an introducer sheath having a reinforced tapered distal end portion.

2. Background Information

Numerous advances of considerable note have occurred in medical surgical techniques over the last few decades. Among the most significant advances has been the adoption, and now-routine performance, of a variety of minimally invasive procedures. Such procedures include angioplasty, endoscopy, laparoscopy, and arthroscopy, as well as numerous other diagnostic and therapeutic operations. These minimally invasive procedures can be distinguished from conventional open surgical procedures in that access to a site of concern within a patient is achieved through a relatively small incision, into which a tubular device (or tubular portion of a device) is inserted or introduced. The tubular device, or device portion, keeps the incision open while permitting access to the target site via the interior (i.e., the lumen) of the tube.

Body passageways in which introducer apparatuses have been used to introduce medical interventional devices and/or liquid medicaments include the esophagus, trachea, colon, biliary tract, urinary tract, and virtually all portions of the vascular system. One particularly significant example of a minimally invasive technique involves the temporary or permanent implantation of a medical interventional device, such as a stent, into a body passageway of a patient. Other examples involve the transmission of a liquid medicament to a target area, and/or the withdrawal of body fluid from the body passageway.

When carrying out these, and other, desired techniques, communication with the passageway is typically attained by inserting an access device, such as an introducer sheath, into the body passageway. One typical procedure for inserting the introducer sheath is the well-known Seldinger percutaneous entry technique. In the Seldinger technique, a needle is initially injected into the passageway, such as a vessel, and a wire guide is inserted into the vessel through a bore of the needle. The needle is withdrawn, and an introducer assembly is inserted over the wire guide into the opening in the vessel.

Typically, the introducer assembly includes an outer introducer sheath, and an inner dilator having a tapered distal end. The tapered end of the dilator stretches the opening in the vessel in controlled fashion, so that introduction of the larger diameter introducer sheath may then be carried out with a minimum of trauma to the patient. In some instances, a small diameter dilator is initially inserted into the opening to stretch the opening to a first diameter, followed sequentially by one or more larger diameter dilators stretching the opening to sequentially larger diameters until an opening of the desired diameter is achieved. Following advancement of the introducer sheath into the opening, the dilator is removed, leaving at least the distal portion of the larger diameter introducer sheath in place in the vessel. The interventional device, such as a stent, etc., or the liquid medicament may then be passed through the introducer sheath for delivery to the target site.

Historically, percutaneous insertion techniques were problematic, due in large part to the lack of flexibility and/or kink resistance of the sheath. Early sheaths were generally formed of a relatively stiff fluoropolymer, such as polytetrafluoroethylene (PTFE) or fluorinated ethylene propylene (FEP). The sheaths were typically of thin-walled construction, and were prone to kinking, particularly when threaded through tortuous pathways within the body. Increasing the thickness of the sheath only minimally improved the kink resistance of the sheath. At the same time, the added thickness occupied valuable space in the vessel, thereby minimizing the diameter of the interventional device that could be passed therethrough. In addition, increasing the thickness of the sheath necessitated the use of a larger entry opening than would otherwise be required.

A kinked sheath is essentially unusable, and generally cannot be straightened while positioned in the body of the patient. Consequently, once a sheath kinks, the sheath must be removed, leaving an enlarged, bleeding opening which typically cannot be reused. Access to the vessel must then be re-initiated at an alternative site, and the process repeated with a new sheath. In many cases, a suitable alternative site is not available, and the percutaneous procedure must be abandoned altogether in favor of a different, and often more intrusive, technique.

In recent years, introducer sheaths have been improved in order to enhance their flexibility and kink resistance. Such sheaths are now routinely used to percutaneously access sites in the patient's anatomy that previously could not be accessed with existing sheaths, or that could be accessed only upon the exercise of an undesirable amount of trial and error, with the concomitant discard of sheaths whose placement had been unsuccessful.

Many newer sheaths exhibit a much higher degree of kink resistance than was achievable with prior art sheaths. One example of a flexible, kink resistant introducer sheath is described in U.S. Pat. No. 5,380,304. The sheath described in this patent includes a lubricious inner liner formed of a relatively stiff fluoropolymer, such as PTFE. A helical coil is fitted over the liner. An outer tube formed of a more flexible polymer, such as a polyether block amide, nylon, or polyurethane, is fitted over the liner and coil. The entire assembly is placed in a heat shrink enclosure, and heated in an oven. The outer surface material melts and bonds to the outer surface of the inner liner through the coil turns.

U.S. Patent Publication No. 2001/0034514 discloses an introducer sheath similar in many respects to the sheath of the '304 patent. The sheath in the patent publication is formed such that the proximal end of the sheath has a higher stiffness, while the distal end has a lower stiffness. Since the distal portion of the sheath has a lower stiffness (and therefore is more flexible) than the proximal portion, the sheath is able to traverse portions of the anatomy that would have been difficult, if not impossible, to traverse with stiffer sheaths. Since the proximal portion has a higher stiffness (and is therefore less flexible) than the distal portion, the sheath maintains the trackability to traverse tortuous areas of the anatomy. This presence of the coil reinforcement also enables this sheath to be kink resistant through a wide range of bending angles. Each of the patent documents cited above is incorporated herein by reference.

The development of introducer sheaths, such as those described above, has revolutionized the practice of medicine. In particular, this development has enhanced the ability of the physician to introduce medical interventional devices and liquid medicaments into target sites that had previously been difficult, if not impossible, to reach without the necessity of carrying out much more intrusive open surgical operations. The percutaneous methods described are generally less expensive than the open surgical methods previously employed, are less traumatic to the patient, and typically require a shorter patient recovery time.

Notwithstanding the benefits that have been achieved by the use of such introducer sheaths, new challenges continue to be faced. For example, as noted above, introducer sheaths are frequently introduced into the body passageway in combination with a tapered inner dilator. In some cases, it is necessary to utilize multiple dilators in order to gradually widen the opening to the desired diameter. The necessity to use multiple dilators adds extra physician time to the procedure, and adds an element of expense that could otherwise be avoided. In addition, in some instances, the seal formed by the introducer in the bodily opening is less secure than desired, thereby allowing seepage of fluids through the opening. Similarly, the inner diameter of the introducer sheath is typically tailored to closely mirror the diameter of the opening. In such instances, the close tolerance between the inner diameter of the sheath and the outer diameter of an interventional device (a wire guide, a medical interventional device such as a stent, etc.) inserted therethrough may result in an undesirable amount of drag on the device as it passes through the inner diameter of the sheath. In still other instances, the column strength and/or the torque of the distal portion of the sheath may not be sufficient to optimize passage, or tracking, of the sheath through the narrow confines of the body.

It is desired to provide an introducer sheath that is structured to overcome the problems of the prior art. More particularly, it is desired to provide a smaller diameter introducer sheath having a reinforced tapered distal end that is capable of exhibiting high column strength for pushability, and high torque strength for control in a manner that is more characteristic of the larger diameter sheaths that are used to reach more readily accessible areas of the vascular system or body. It is desired that the smaller diameter sheath terminate in a much smaller diameter distal end to enable the sheath to reach more confined or occluded areas that would not otherwise be possible with a larger diameter sheath.

### BRIEF SUMMARY

The present invention addresses the shortcomings in the prior art. In one form thereof, the invention comprises an introducer sheath comprising an inner liner having a proximal end and a distal end. The inner liner has a first length comprising a major diameter, a second length comprising a minor diameter, and a third length axially disposed between the first and second lengths. The third length comprises a transition between the major and minor diameters. The proximal end comprises the major diameter, and the distal end comprises the minor diameter. The inner liner may comprise a heat shrinkable fluoropolymer, such as PTFE. A reinforcing member, such as a helical coil, is positioned over the inner liner and spans at least a portion of the first, second, and third lengths. An outer jacket having a proximal end and a distal end is positioned longitudinally around the reinforcing member and inner liner. The outer jacket has a first length comprising a major diameter, a second length comprising a minor diameter, and a third length axially disposed between the first and second lengths. The third length comprises a transition between the outer jacket major and minor diameters. The outer diameter first, second, and third lengths substantially correspond to and are disposed radially outwardly of the respective inner liner first, second, and third lengths.

In another form thereof, the invention comprises an introducer sheath comprising an inner liner having a larger diameter proximal portion, and a tapered portion distal of the larger diameter proximal portion. A reinforcing member, such as a coil, is positioned over the inner liner and spans at least a portion of each of the inner liner proximal portion and the tapered portion. The reinforcing member has a taper corresponding to at least a portion of the taper of the inner liner tapered portion. An outer jacket is positioned longitudinally around the reinforcing member and the inner liner. The outer jacket has a larger diameter proximal portion and a tapered portion distal of the larger diameter proximal portion. The outer jacket is bonded to an outer surface of the inner liner.

In still another form thereof, the invention comprises a method of forming an introducer sheath. An inner liner having a proximal end and a distal end is provided. The inner liner has a first length of a major diameter, a second length of a minor diameter, and a third length axially disposed between the first and second lengths. The third length comprises a transition between the inner liner major and minor diameters. The proximal end comprises the inner liner major diameter, and the distal end comprises the inner liner minor diameter. A reinforcing member is positioned over the inner liner, wherein the reinforcing member spans at least a portion of each of the first length, second length, and third length. An outer jacket is positioned over the reinforcing member and the inner liner. The outer jacket has a proximal end and a distal end, and has a first length of a major diameter, a second length of a minor diameter, and a third length axially disposed between the first and second lengths. The third length comprises a transition between the outer jacket major and minor diameters. The proximal end comprises the outer jacket major diameter, and the distal end comprises the outer jacket minor diameter. The outer jacket is bonded to an outer surface of said inner liner.

### BRIEF DESCRIPTION OF THE DRAWINGS

FIG. 1 is a side view of an introducer sheath formed according to an embodiment of the present invention;

FIG. 2 is a side view of a sheath inner liner positioned over a mandrel;

FIG. 3 is a side view of the inner liner and mandrel as shown in FIG. 2, with a coil fitted over the inner liner;

FIG. 4 is a partially sectioned longitudinal view of the inner liner and mandrel as shown in FIG. 3, wherein an outer jacket has been positioned over the sheath and coil, and the entire assembly is enclosed in a heat shrink layer;

FIG. 5 is a partially sectioned longitudinal view of the assembly of FIG. 4, following melting of the outer jacket and removal of the heath shrink layer; and

FIG. 6 is a side view of the resulting sheath, partially in section, following removal of the mandrel.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated apparatus, and such further applications of the principles of the invention as illustrated therein being contemplated as would normally occur to one skilled in the art to which the invention relates.

In the following discussion, the terms “proximal” and “distal” will be used to describe the opposing axial ends of the inventive sheath, as well as the axial ends of various component features. The term “proximal” is used in its conventional sense to refer to the end of the sheath (or component thereof) that is closest to the operator during use of the sheath. The term “distal” is used in its conventional sense to refer to the end of the sheath (or component thereof) that is initially inserted into the patient, or that is closest to the patient during use.

It is well-known to form an introducer sheath by heat shrinking an outer polymeric jacket onto an inner liner formed of a lubricious material, such as a fluoropolymer. Sheaths formed by this process typically include a constant diameter reinforcing member, such as a coil and or a braid, positioned between the outer polymeric jacket and the inner liner. In these cases, the outer polymeric jacket is generally heat shrunk onto the outer surface of the inner liner through the turns of the coil, or the filaments of the braid. The incorporated-by-reference U.S. patent documents describe and illustrate representative examples of such sheaths.

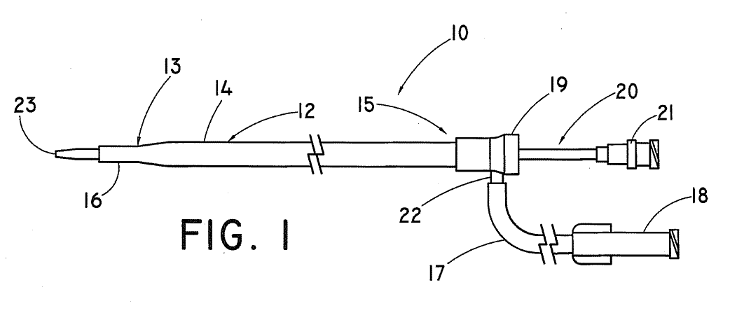

FIG. 1 depicts a side view of an introducer sheath 10 formed according to an embodiment of the present invention. The introducer sheath 10 in this figure includes an outer jacket 12 having a first, or major, diameter 14 along the majority of its length. The major diameter tapers to a second, or minor, diameter 16 by way of tapered portion 13. A conventional connector valve housing 19 may be attached about proximal end 15 of the sheath. Valve housing 19 may include one or more conventional valve disks (not shown), or other conventional valve members for preventing the backflow of fluids therethrough. Housing 19 may also include a side arm 22 to which an extension tube 17 and connector member 18 may be connected in well known fashion for introducing and/or aspirating fluids therethrough.

In FIG. 1, a dilator 20 is shown extending longitudinally through the passageway of introducer sheath 10. Dilator 20 includes a proximal end 21 and a tapered distal end 23 sized and shaped for accessing and dilating a vascular access site over a well-known and commercially available wire guide (not shown). The wire guide may be inserted in the vessel with an introducer needle utilizing, for example, the well-known Seldinger percutaneous access technique.

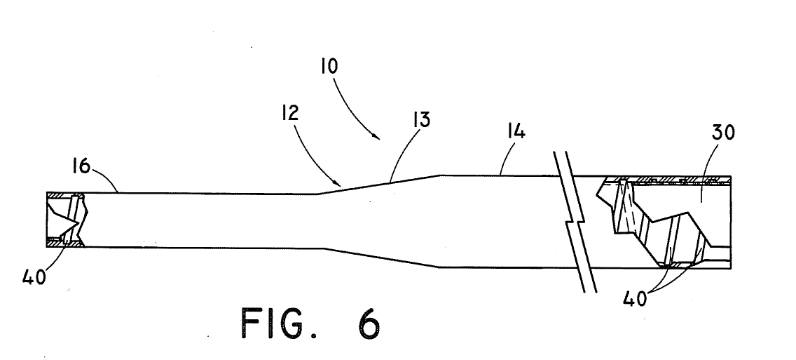

FIG. 6 illustrates a longitudinal view of introducer sheath 10, partially in section. The dilator and connector valve housing have been omitted in this figure. As illustrated, sheath 10 comprises an inner liner 30 formed of a lubricious fluoropolymer, such as polytetrafluoroethylene (PTFE) or fluorinated ethylene propylene (FEP). A reinforcing member 40, in this case a helical coil, is wound or otherwise fitted over the outer surface of the inner liner 30. The outer jacket 12 is fitted over the coil 40 and the inner liner 30. Further details regarding the inner liner, reinforcing member, and outer jacket are provided below.

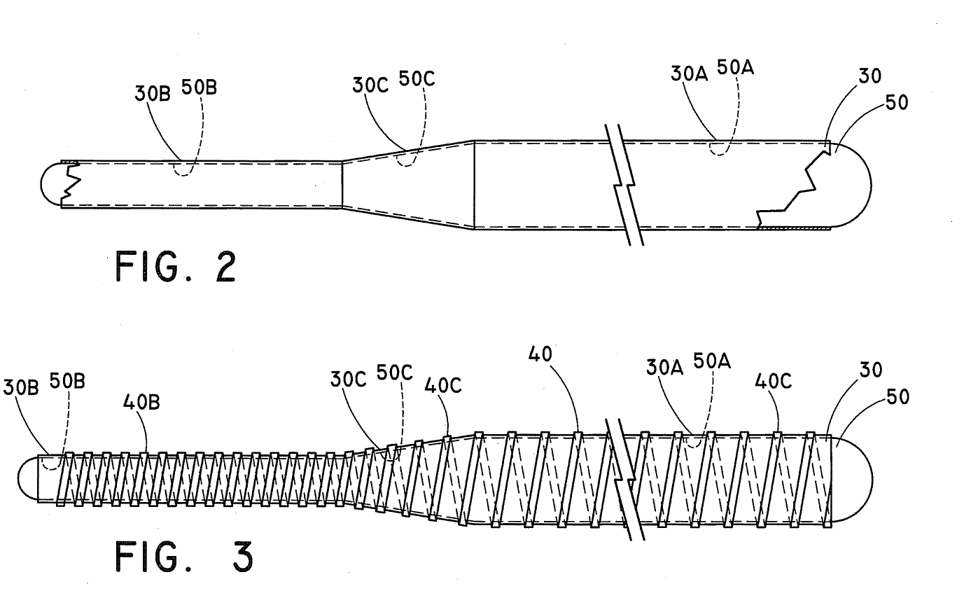

FIGS. 2-5 illustrate further details of introducer sheath 10, and describe a preferred method of forming the introducer sheath. Those skilled in the art will appreciate that many other techniques may be suitable for forming the sheath, such as the techniques described in the incorporated-by-reference patent documents, as well as other techniques known in the art. In the preferred embodiment described herein, inner liner 30 comprises a heat shrinkable fluoropolymer tube. Preferably, the heat shrinkable fluoropolymer comprises PTFE.

The use of the heat shrinkable tube for the inner liner enables the liner to shrink upon application of heat to conform to the size and shape of a substrate. Suitable techniques for forming heat shrinkable tubing are known in the art. For example, heat shrinkable PTFE tubing may be formed by radially expanding, or stretching, a tubular length of PTFE to an inner diameter of, for example, about two to four times the inner diameter of the original PTFE tube. The radially stretched tube is then placed on a mandrel and exposed to heat and pressure, whereupon the radially stretched tube shrinks to assume the dimensions of the underlying mandrel. Upon cooling of the tube and removal of the mandrel, the inner diameter of the PTFE tube retains the outer diameter of the mandrel.

Heat shrink fluoropolymers are capable of shrinking to recover their original diameter, or alternatively, the diameter of an underlying substrate (such as a smaller diameter mandrel), upon exposure to heat. Those skilled in the art can determine an appropriate amount of heat and pressure necessary to shrink a particular fluoropolymer, taking into consideration such factors as the melting point of the fluoropolymer, thickness and configuration of the tube, etc. Most fluoropolymers, however, have a shrinkage temperature within a general range for that particular fluoropolymer. Heat shrinkable PTFE, for example, typically shrinks at a temperature of about 650° F. (340° C.).

In addition to forming heat shrinkable fluoropolymer tubing by methods such as that described above, suitable heat shrinkable tubing may also be obtained from commercial sources. One such source is Zeus Industrial Products, Inc., of Orangeburg, S.C. Commercial vendors are typically capable of providing fluoropolymer heat shrink tubing in a variety of lengths, thicknesses and diameters, which tubing has a shrink temperature appropriate for the intended use.

FIG. 2 is a side view illustrating a PTFE inner liner 30 that has been heat shrunk over a mandrel 50. Mandrel 50 is typically formed of a material, such as stainless steel, that is capable of withstanding the temperatures utilized for shrinking inner liner 30. Mandrel 50 is machined or otherwise formed to have the same general configuration as desired in the introducer sheath. As illustrated, mandrel 50 has respective major and minor diameter segments 50A and 50B, respectively, and a transition 50C between these segments. Preferably, the outer surface of the stainless steel mandrel is ground, laser cut, or otherwise treated to have a very smooth surface finish.

A segment of heat shrinkable PTFE liner 30 is selected having a recovered inner diameter slightly smaller than the diameter of minor segment 50B of the mandrel, and an expanded diameter slightly larger than the diameter of major diameter 50A of the mandrel. Utilizing a liner having a slightly smaller recovered diameter avoids wrinkles or folds in the inner diameter of the finished part. When selecting the dimensions of the PTFE liner segment, consideration should also be given for shrinkage in length, as well as diameter, of the polymeric liner. Therefore, the length of the PTFE liner should be longer than the longest length desired of the finished product, and the liner should be positioned over the mandrel to provide an excess length at each end.

Once the liner is positioned over the mandrel, the liner and mandrel are exposed to heat to shrink the PTFE liner so that it conforms to the outer diameter of the mandrel along the length of the mandrel. This can be accomplished in an oven at or above the transition temperature for the heat shrinkable tubing. When inner liner 30 is heat shrinkable PTFE, the tube and mandrel will typically be exposed to a temperature of at least about 650° F. (340° C.) in order to carry out the heat shrink. One particularly preferred heating means utilizes a commercially available shrinking system, such as the Shrink Cycler (Model 810-A), available from Beahm Designs, of Campbell, Calif. Commercially available systems, such as the Shrink Cycler, are commonly used when applying heat shrink tubing over substrates of various lengths. Those skilled in the art will appreciate that other heating means may be substituted.

Following the heat shrink, PTFE liner 30 has a major diameter segment 30A and a minor diameter segment 30B, with a transition 30C therebetween. The use of the lubricious PTFE as the inner liner material provides a slippery inner surface to the sheath, thereby allowing easy insertion and withdrawal of the medical interventional device passed therethrough. The wall of the inner liner 30 has sufficient radial rigidity to prevent a reinforcing member, such as coil 40, from protruding into the inner sheath passageway. Preferably, the outer surface of inner liner 30 is roughened by chemical etching or other conventional procedures to enhance the strength of the bond with the outer jacket 12, to be described.

Although the PTFE inner liner is preferably formed from a heat shrinkable material as described, other alternatives are also possible. For example, if desired, liner 30 can be formed from separate tubular members cut to the desired diameters and configurations (e.g., taper) of respective segments 30A, 30B and 30C, and assembled to form a liner having the shape illustrated in FIG. 2.

In FIG. 3 a coil 40 has been slid or otherwise transferred onto the outer surface of the heat shrunk PTFE inner liner 30. Those skilled in the art will appreciate that there are many alternative ways of applying a coil over the inner liner that may be suitable for a particular instance. For example, if the outer diameter of the inner liner does not vary to a significant degree between segments 30A and 30B, an initially constant diameter coil can be wound over the inner liner, e.g., utilizing a conventional winding machine of the type well known in the art. Alternatively, a coil pre-formed with varying diameters (coinciding substantially with the diameters of inner liner segments 30A, 30B and transition 30C) can be slid over the inner liner. As still another variation, separate coil segments 40A, 40B, 40C, as described below, can be slid over the inner liner, and aligned in appropriate manner.

Coil 40 preferably has a major diameter segment 40A, a minor diameter segment 40B, and a transition 40C therebetween. The respective inner diameters of segments 40A, 40B, and transition 40C should closely match the outer diameter of corresponding segment 30A, 30B and transition 30C of inner liner 30 in order to minimize the diameter of the resulting sheath 10. Preferably, coil 40 will extend substantially the entire length of the inner liner. If desired, however, an uncoiled segment may be left at either or both ends. Typically, the uncoiled portion will not exceed more than a few cm at each end (e.g. 2-5 cm), however, a larger uncoiled portion at either, or both, ends may be provided if desired. An uncoiled portion may be provided, e.g., to facilitate flaring of the proximal sheath end, or to add flexibility to a distal tip. As a further alternative, a coil may be omitted altogether from any discrete portion of the sheath if desired.

Coil reinforcing members for introducer sheaths are well known in the art, and coil 40 can be formed of any typical composition (e.g., metals or metal alloys), and can be formed to have virtually any cross-sectional shape, thickness, pitch, compression, etc., as may be desired. Preferably, in order to minimize the diameter of the resulting sheath, a flat wire coil is utilized. Typically, the various turns of the coil 40 will have a generally constant pitch, and have generally uniform spacing between adjacent coil turns.

In an alternative embodiment, the reinforcing member may comprise other conventional reinforcing members known in the medical arts, such as a woven braid. Braids are also well known reinforcing members used in medical devices, and those skilled in the art can readily fashion a braid appropriate for use herein. As a still further alternative, sheath 10 may include a coil along part of its length, and a braid along another part of the length.

As best shown in FIG. 4, the tubular outer jacket 12 is then slid or otherwise positioned over inner liner 30 and coil 40. Outer jacket 12 may be formed of any well-known polymer commonly used for such purpose. Preferably, outer jacket 12 comprises a heat formable polyamide material, such as nylon, or a polyether block amide (PEBA). This heat formable material melts upon heating, such that portions flow between the respective turns of the coil, and bond to the roughened outer surface of the inner liner.

In a preferred embodiment, outer jacket 12 comprises a length of bump tubing that is sized and shaped to closely fit over the coil and liner assembly of FIG. 3, with little clearance between the inner surface of the bump tube (i.e., outer jacket 12), and the coil and inner liner. “Bump tubing” is a term of art used in the tubing industry to refer to a tubular element wherein both the inner and outer diameters of the tube vary along the length of the tubular element. Bump tubing may be formed utilizing conventional procedures to have virtually any arrangement of diameters as may be desired for a particular use. Extrusion is a common way of forming bump tubes in the medical arts. Bump tubing may be obtained from a variety of commercial sources, such as Zeus Industrial Products, Inc., and can generally be made to any specification appropriate for use in the sheath herein.

In FIG. 4, outer jacket 12 includes a major diameter segment 12A, a minor diameter segment 12B, and a transition 12C therebetween. Typically, a bump tube is formed (e.g., extruded) to the desired size and shape, which in this case, closely approximates the shape of inner liner 30, but has a slightly larger inner diameter than the corresponding features on the outer diameter of the inner liner.

As an alternative to the use of bump tubing, separate lengths of tubing corresponding to respective segments 12A, 12B and transition 12C can be utilized. In this instance, a tapered transition portion may be slid onto the smaller diameter end of the liner until it is placed over the transition portion 30C of the inner liner. Respective larger diameter and smaller diameter segments 12A and 12B may then be slid onto the respective major and minor diameter ends 30A and 30B of the inner liner. If desired, the wall thicknesses and materials of respective segments may be varied to achieve any desired characteristics of a particular segment.

A heat shrink enclosure 54 is then slid over the assembly, as also shown in FIG. 4. The heat shrink enclosure 54 is typically formed of a polymeric material, such as FEP, that is capable of being heated to a temperature in excess of the melt temperature of the outerjacket 12 (about 364° F. (185° C.). The enclosure 54 with the assembly comprising the inner liner 30, coil 40, and outer jacket 12 positioned therein is then placed in an oven, Shrink Cycler, or other heat source, and heated to the melting temperature of the outer jacket.

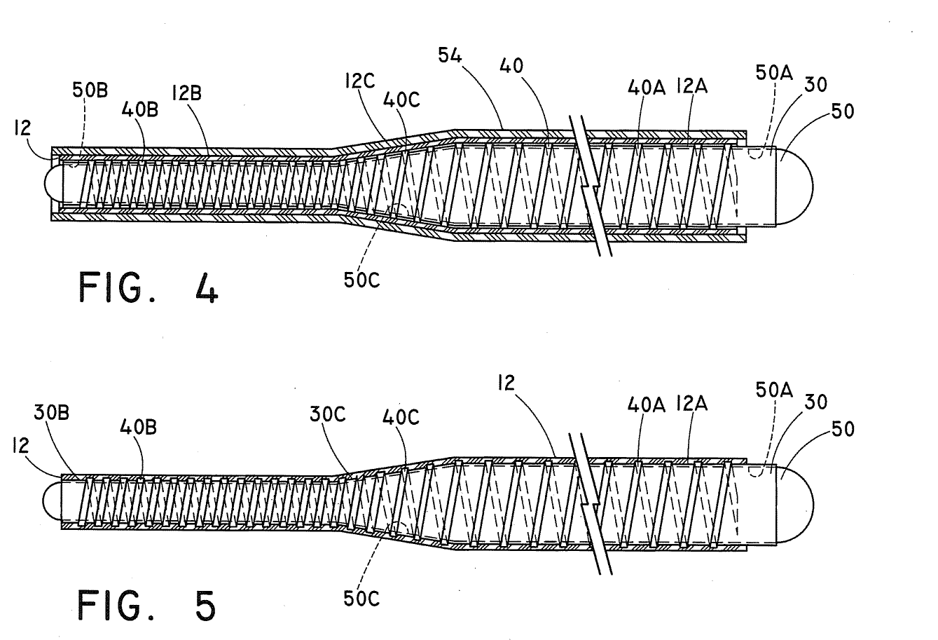

As the heat shrink enclosure shrinks upon the application of heat, the heated outer jacket 12 melts and is compressed by the shrinking FEP enclosure between the turns of the coil 40 to mechanically bond with the roughened surface of inner liner 30. When the outer jacket is formed from multiple pieces of tubing as described above, the respective tubing pieces also bond to each other. Following heating, the contents are removed from the oven, and allowed to cool. The heat shrink tube is then split from the exterior of the sheath and removed. This is shown in FIG. 5. The mandrel is slid out from the interior of the sheath. The respective longitudinal ends of the sheath may be trimmed to a desired length.

The dimensions (e.g., length, inner and outer diameters, wall thickness, etc.) of the various elements mentioned above should be selected in view of the proposed use of the introducer sheath 10. Those skilled in the art will appreciate that the dimensions for a particular sheath will be governed in large part on the proposed use for that sheath. Thus, for example, for a proposed neurovascular application, the sheath may have a first length (proximal end) of a very small French size, on the order of about 4 French (1.35 mm), that tapers down to about 2 French (0.67 mm) at the second length (distal end). On the other hand, for use in larger arteries, the sheath can have a much larger diameter, on the order of about 8 French (2.7 mm) or even larger at the proximal end, that tapers down to a distal end having a diameter of about one-half of the diameter of the proximal end.

The differences between the respective diameters of the proximal end and the distal end need not necessarily differ by a factor of two as described in these examples, and a greater, or lesser difference may be appropriate for a particular case. All dimensions provided herein are only intended to be examples of possible dimensions that can be selected for a particular use, and it is believed that those skilled in the art can readily optimize dimensions for a particular use once benefit of the present disclosure is had. While a modest amount of trial-and-error may be needed to obtain optimal dimensions, it is believed that any required experimentation will not be undue.

The respective lengths of inner liner 30 and outer jacket 12 on the finished sheath will generally be the same. The length of the major diameter segments 12A, 30A will typically comprise approximately 70% of the length of the completed sheath 10, and more typically, at least about 90% or more of the length of sheath 10. Those skilled in the art will appreciate that these dimensions are also only intended to be examples of possible dimensions that may be selected by one skilled in the art, and that the actual dimensions for a particular sheath may be varied depending upon the intended use of the sheath.

As a further variation of the preferred embodiments described, not all embodiments need include a separate minor diameter segment, as designated in the figures and discussion hereinabove as segments “B”. Rather, in these embodiments, the sheath merely includes segments “A” and “C”, wherein “C” tapers to a sheath distal end. In this case, coil 40 preferably extends at least substantially to the distal end of the sheath. As a still further variation, the coil may extend through the taper, and then be joined by a smaller diameter distal tip. The distal tip may be formed of a composition different from that of the proximal end. In this case, the distal tip composition will generally be more flexible than the composition of the proximal end. The small diameter tip may, or may not, include a coiled portion.

Those skilled in the art will appreciate that other modifications may be made to the sheath as described herein for a particular purpose. For example, the sheath can be formed to include one or more segments along its length that differ in flexibility from other segments. Typically, this is accomplished by forming the outer jacket of one or more segments of different durometer, generally aligned in order of decreasing durometer (e.g., increased flexibility) from the proximal end to the distal end of the sheath. Alternatively, this can be accomplished by continuous extrusion of segments of different durometer and/or thickness. Routine additional features, such as hydrophilic coatings, radiopaque markers, side ports, etc., that are well know for use with introducer sheaths may be incorporated in well known fashion.

Additional features of the construction or composition of the various elements of the introducer sheath 10 not otherwise discussed herein are not believed to be critical to the present invention, so long as the recited elements possess the strength and/or mechanical properties needed for them to perform as desired. Many such details not described herein are recited in detail in the incorporated-by-reference patent documents. Additional details of construction are believed to be well within the ability of one of ordinary skill in the art.

It is therefore intended that the foregoing detailed description be regarded as illustrative rather than limiting, and that it be understood that it is the following claims, including all equivalents, that are intended to define the spirit and scope of this invention.

1. An introducer sheath, comprising:
an inner liner having a proximal end and a distal end, said inner liner having a first length comprising a major diameter, a second length comprising a minor diameter, and a third length axially disposed between said first and second lengths, said third length comprising a transition between said major and minor diameters, said proximal end comprising said major diameter, and said distal end comprising said minor diameter;
a reinforcing member positioned over said inner liner and spanning at least a portion of each of said first length, second length, and third length; and
an outer jacket positioned longitudinally around said reinforcing member and said inner liner, said outer jacket having a proximal end and a distal end, said outer jacket having a first length comprising a major diameter, a second length comprising a minor diameter, and a third length axially disposed between said first and second lengths, said third length comprising a transition between said outer jacket major and minor diameters, said outer diameter first, second, and third lengths substantially corresponding to and disposed radially outwardly of said respective inner liner first, second, and third lengths.

2. The introducer sheath of claim 1, wherein said reinforcing member comprises a coil having a plurality of coil turns, and wherein said outer jacket is bonded to an outer surface of said inner liner between said coil turns.

3. The introducer sheath of claim 1, wherein said inner liner comprises a fluoropolymer, and said reinforcing member comprises a flat wire coil.

4. The introducer sheath of claim 3, wherein said inner liner comprises heat shrinkable PTFE.

5. The introducer sheath of claim 1, wherein said outer jacket comprises bump tubing.

6. An introducer sheath, comprising:
an inner liner, said inner liner having a larger diameter proximal portion, and a tapered portion distal of said larger diameter proximal portion;
a reinforcing member positioned over said inner liner and spanning at least a portion of each of said inner liner proximal portion and tapered portion, said reinforcing member having a taper corresponding to at least a portion of said taper of said inner liner tapered portion; and
an outer jacket positioned longitudinally around said reinforcing member and said inner liner, said outer jacket having a larger diameter proximal portion and a tapered portion distal of said larger diameter proximal portion, said outer jacket bonded to an outer surface of said inner liner.

7. The introducer sheath of claim 6, wherein said reinforcing member comprises a coil, and said outer jacket is bonded to said outer surface through respective turns of said coil.

8. The introducer sheath of claim 7, said inner liner further comprising a smaller diameter portion distal of said inner liner tapered portion, and said outer jacket further comprising a smaller diameter portion distal of said outer jacket tapered portion.

9. The introducer sheath of claim 8, wherein said coil extends over at least a portion of said inner liner smaller diameter portion.

10. The introducer sheath of claim 6, wherein said inner liner comprises PTFE, and said reinforcing member comprises a coil

11. The introducer sheath of claim 10, wherein said inner liner comprises heat shrinkable PTFE, and said reinforcing member comprises a flat wire coil.

12. The introducer sheath of claim 6, wherein said outer jacket comprises bump tubing.

13. A method of forming an introducer sheath, comprising:
providing an inner liner having a proximal end and a distal end, said inner liner having a first length of a major diameter, a second length of a minor diameter, and a third length axially disposed between said first and second lengths, said third length comprising a transition between said inner liner major and minor diameters, said proximal end comprising said inner liner major diameter, and said distal end comprising said inner liner minor diameter;
positioning a reinforcing member over said inner liner, said reinforcing member spanning at least a portion of each of said first length, second length, and third length;
positioning an outer jacket over the reinforcing member and the inner liner, said outer jacket having a proximal end and a distal end, said outer jacket having a first length of a major diameter, a second length of a minor diameter, and a third length axially disposed between said first and second lengths, said third length comprising a transition between said outer jacket major and minor diameters, said proximal end comprising said outer jacket major diameter, and said distal end comprising said outer jacket minor diameter; and
bonding said outer jacket to an outer surface of said inner liner.

14. The method of claim 13, wherein said inner liner comprises a heat shrinkable fluoropolymer, and said inner liner first, second and third lengths are formed by placing said heat shrinkable fluoropolymer over a shaped mandrel, and exposing said inner liner to heat.

15. The introducer sheath of claim 14, wherein said fluoropolymer comprises PTFE.

16. The introducer sheath of claim 15, wherein said reinforcing member comprises a coil.

17. The method of claim 16, wherein said outer jacket is bonded to said inner liner surface by heat shrinking an assembly comprising said inner liner, reinforcing member, and outer jacket.

18. The method of claim 17, wherein said outer jacket first, second, and third lengths are initially separate, said lengths being sequentially aligned over the reinforcing member and the inner liner, said initially separate lengths being bonded during said heat shrinking of said assembly.

19. The method of claim 13, wherein said outer jacket comprises bump tubing shaped to define said major diameter, minor diameter, and transition between said outer jacket major and minor diameters

20. The method of claim 13, wherein said inner liner first, second, and third lengths are initially separate, said lengths being sequentially aligned over a mandrel.
